(19) United States
(12) Patent Application Publication
(10) Pub. No.: US 2017/0087000 A1
(43) Pub. Date: Mar. 30, 2017

(54) REHABILITATION DEVICE FOR A DAMAGED OR SURGICALLY REPAIRED KNEE

(71) Applicant: Frank J. Cain, Naples, FL (US)

(72) Inventor: Frank J. Cain, Naples, FL (US)

(21) Appl. No.: 15/274,857

(22) Filed: Sep. 23, 2016

Related U.S. Application Data
(60) Provisional application No. 62/232,823, filed on Sep. 25, 2015.

(51) Int. Cl. A61F 5/01; A61F 7/02

(52) U.S. Cl. CPC A61F 5/0123; A61F 5/013; A61F 5/0127; A61F 7/02; A61F 2005/0165

(57) ABSTRACT

Systems and devices of providing therapy to injured joints are disclosed. The device comprises a first body coupling member and a second body coupling member, wherein the first and second body coupling members are adapted to surround a patient's joint; a cage coupling the first body coupling member to the second body coupling member, wherein the cage is adapted to traverse the joint; at least one elevating support coupled to the first body coupling member; at least one elevating support coupled to the second body coupling member; at least one clip coupled to the first body coupling member adapted to be coupled to a therapy device; and at least one clip coupled to the second body coupling member adapted to be coupled to the therapy device.

### REFERENCE TO RELATED APPLICATIONS

The present application claims priority to Provisional U.S. Application No. 62/232,823, filed Sep. 25, 2015, entitled “REHABILITATION DEVICE FOR A DAMAGED OR SURGICALLY REPAIRED KNEE,” and is incorporated herein in its entirety.

### BACKGROUND

1. Field of the Invention

The invention is directed to a medical brace. More specifically, the invention is directed to a medical brace that incorporates elevating supports.

2. Background of the Invention

It is accepted professional medical philosophy that the human knee requires a prolonged period of rehabilitation when damaged or subject to arthroscopic or replacement surgery. The prognosis for recovery is enhanced when the knee is elevated above the heart for draining of entrapped fluids while the patient is lying down and when the swelling and muscle contractions are subject to chilling. The mantra of “elevate and ice” is basic to joint recovery strategy.

Damage knees, elbows, ankles, wrists, shoulders, hips, and other joints, as well as joints subject to surgery, generally swell with entrapped bodily fluids and muscle expansion intended to cushion the damaged areas and to protect from pain and further trauma. Rehabilitation techniques are intended to reduce the swelling, pain and fluid buildup by mild and increasing exercise, movement, massage and cooling. Following rehabilitation physical therapy, it is advisable to elevate the damage joint slightly above the heart while lying prone to assist draining entrapped fluids and to apply semi frozen gel packs, moistened frozen small towels, or even packages of frozen peas or other small vegetables.

An injury, such as a knee or ankle sprain, pain and swelling, can be relieved and promote healing and flexibility can be promoted with “R.I.C.E.” or Rest, Ice, Compression, and Elevation. Rest includes resting and protecting the injured or sore area. For example, a person should stop, change, or take a break from any activity that may be causing pain or soreness. Ice involves applying an ice or cold pack right away to prevent or minimize swelling since cold will reduce pain and swelling. After 48 to 72 hours, if swelling is gone, application of heat to the area that is hurt may be beneficial. Compression, or wrapping the injured or sore area with an elastic bandage (such as an Ace wrap), will help decrease swelling. Elevating the injured or sore area while applying ice additionally helps minimize swelling, especially if elevated above the heart.

### SUMMARY OF THE INVENTION

The present invention overcomes the problems and disadvantages associated with current strategies and designs and provides new devices and methods for treating injured joints.

An embodiment of the invention is directed to a brace. The brace comprises a first body coupling member and a second body coupling member, wherein the first and second body coupling members are adapted to surround a patient's joint; a cage coupling the first body coupling member to the second body coupling member, wherein the cage is adapted to traverse the joint; at least one elevating support coupled to the first body coupling member; at least one elevating support coupled to the second body coupling member; at least one clip coupled to the first body coupling member adapted to be coupled to a therapy device; and at least one clip coupled to the second body coupling member adapted to be coupled to the therapy device.

Preferably, the first and second body coupling members are saddle shaped plates securable to a patient with straps. The first body coupling member is preferably placed on a first side of the joint and the second body coupling member is preferably placed on a second side of the joint. In a preferred embodiment, the brace is adapted to promote the healing of a knee, an elbow, a wrist, or an ankle.

The cage is preferably comprised of at least one spine and a pair of hinges. Each spine is preferably comprised of at least one of two coaxial tubes or two parallel slides that accommodate movement of the patient. Preferably, the spines surround the therapy device. In a preferred embodiment, the hinges permit a specified range of motion of the joint.

Preferably, each elevating support is moveable from a closed position to an open position. Preferably, each elevating support is one of a swing-down arm or a telescoping arm. The elevating support are preferably one of adjustable or fixed. In a preferred embodiment, the elevating support is adapted to raise the joint above the patient's heart while the patient is prone. Preferably, the therapy device is a heating device or a cooling device. Preferably, the patient can transition between walking and laying prone with the brace elevating the patient's joint without removing the brace.

Other embodiments and advantages of the invention are set forth in part in the description, which follows, and in part, may be obvious from this description, or may be learned from the practice of the invention.

### DESCRIPTION OF THE DRAWING

The invention is described in greater detail by way of example only and with reference to the attached drawing, in which:

FIG. 1 is a perspective view of an embodiment of the brace on a straight knee.

FIG. 2 is a perspective view of an embodiment of the brace on a bent knee.

FIG. 3 is a side view of an embodiment of the brace.

FIG. 4 is a front view of an embodiment of the brace.

FIG. 5 is a top view of an embodiment of the brace.

FIG. 6 is a perspective view of another embodiment of the brace.

### DESCRIPTION OF THE INVENTION

As embodied and broadly described herein, the disclosures herein provide detailed embodiments of the invention. However, the disclosed embodiments are merely exemplary of the invention that may be embodied in various and alternative forms. Therefore, there is no intent that specific structural and functional details should be limiting, but rather the intention is that they provide a basis for the claims and as a representative basis for teaching one skilled in the art to variously employ the present invention

A problem in the art capable of being solved by the embodiments of the present invention is existing medical braces either do not provide an elevating mechanism and/or do not allow a patient the ability to walk comfortably and to return to the prone position. It has been surprisingly discovered that a device with integrated elevating supports and ice pack holders can permit continuous physical therapy to a damaged or surgically corrected joint as opposed to the need of the patient to frequently replace an ice pack that inhibits movement and potentially cuts off circulation. The device preferably permits the patient to walk normally while chilling the affected joint and to ascend and descend stairs without removing the device. Preferably, the patient can then return to the prone position without removing the device or the chilling pack.

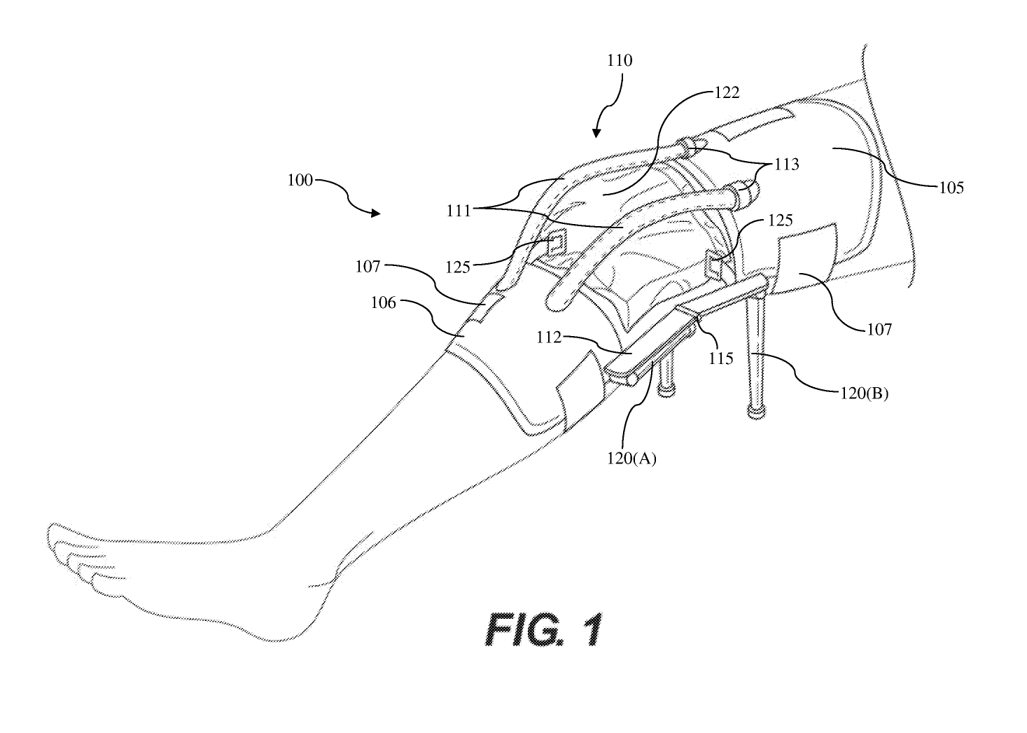

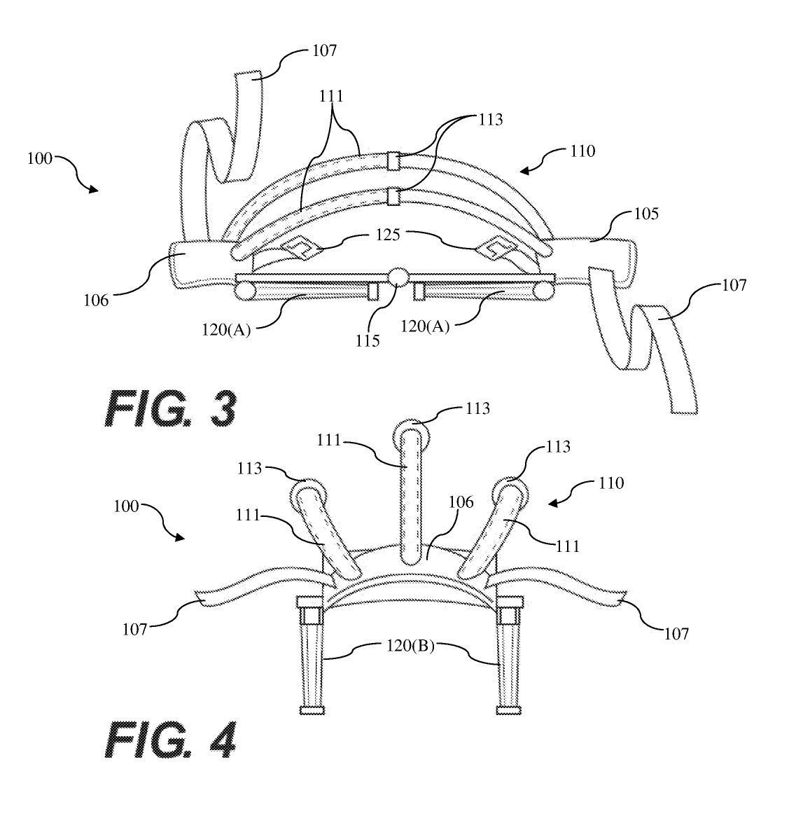

FIGS. 1-5 depict various views of a brace 100. The device is intended to provide a wearable rehabilitation aid that preferably permits a patient to both elevate and chill a damaged or surgically repaired joint. While the invention is described for use on a knee, the brace can be adapted for use on other joints, including but not limited to ankles, elbows, wrists, shoulders, and hips. Additionally, while the invention is described for use on humans, it may be usable on other animals. Brace 100 is preferably constructed of plastic or carbon fiber, is intended to provide a recovery patient with the ease of elevating a joint while lying down and to apply an ice or gel pack or other chilling media while also allowing the patient to stand up, walk for prolonged periods and then returning to the prone position without removing brace. While the invention is described using an ice or gel pack, the patient can use frozen vegetables, instant cold packs, a bag or other container of ice, a microwavable heat pack, an instant heat pack, a hot water container, or another cooling or heating device.

Preferably, brace 100 is comprised of an upper plate or saddle 105 and a lower plate or saddle 106 that when properly positioned straddles the joint. For example, as shown in FIG. 1, upper saddle 105 is positioned on the patient's thigh while lower saddle 106 is positioned on the patient's shin. While saddles are the preferred embodiment, brace 100 may alternatively use straps, cuffs, sleeves, wraps, or other devices to secure brace 100 to the patient. Preferably, upper saddle 105 and lower saddle 106 are held securely to the patient via straps 107. Preferably, straps 107 are hook and loop type devices to fit various sized patients and joints. Other fasteners can be employed as well, for example, ties, clasps, elastic bands, zippers, buttons, snaps, and toggles. Preferably, saddles 106 and 107 are stiff components comprised of, for example, plastic, carbon fiber, metal, wood, or another manmade or naturally occurring material. However, saddles 106 and 107 may be made of flexible materials such as, for example, cotton, wool, neoprene, nylon, mesh, or another manmade or naturally occurring material. While brace 100 is described as having an upper and lower saddle, the two saddles may be interchangeable, thereby allowing the device to be positioned in any orientation.

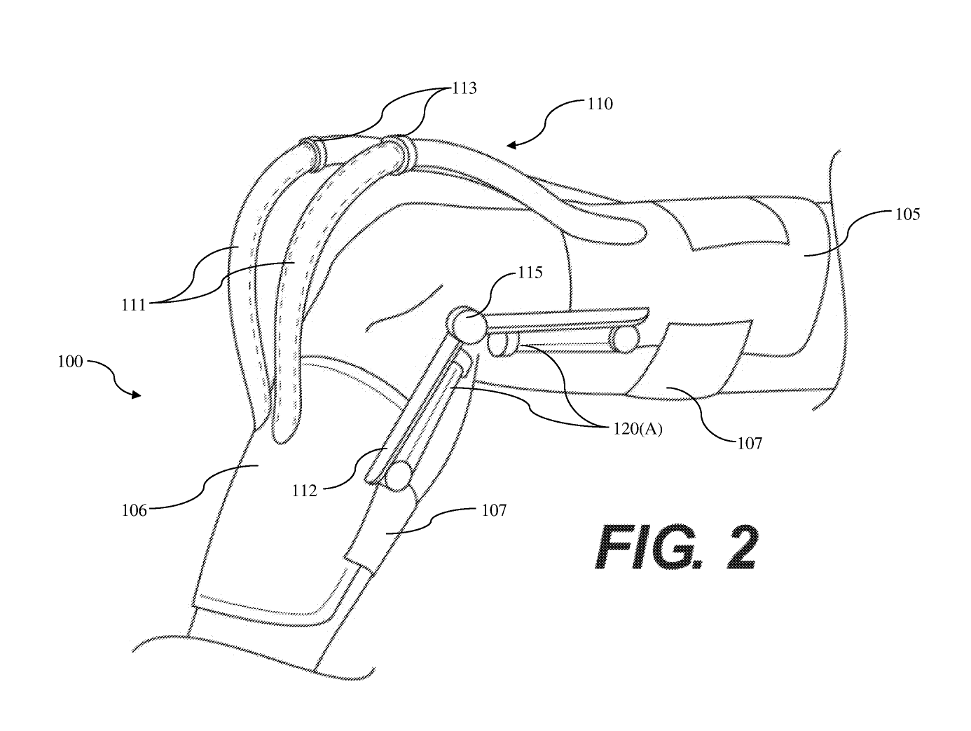

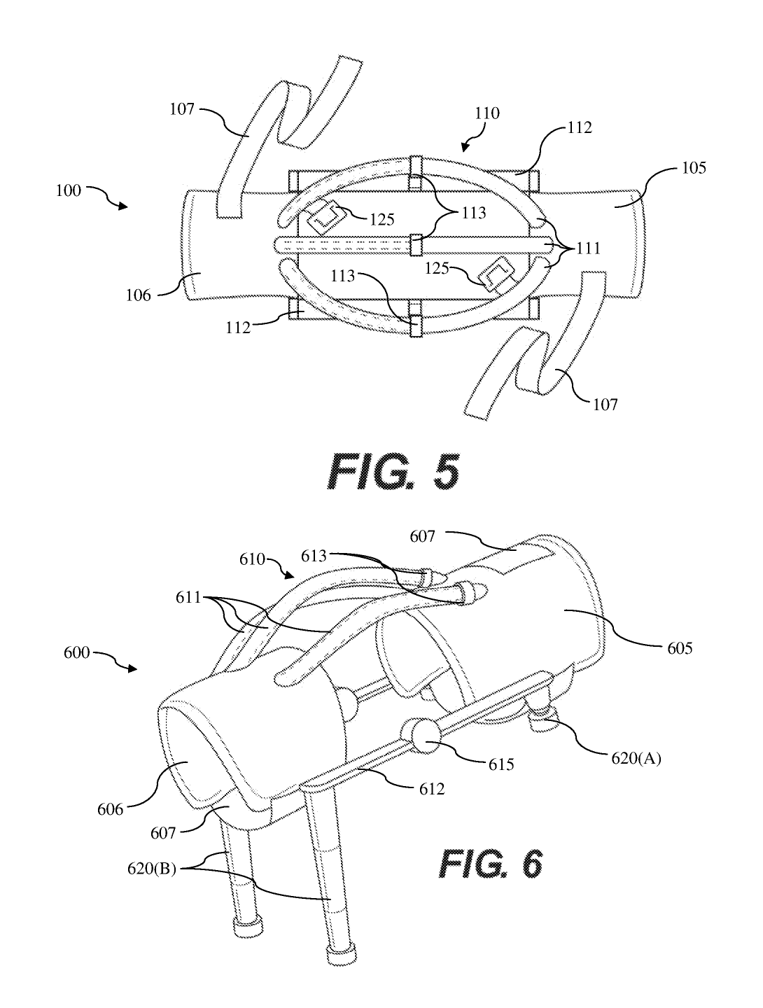

Bridging saddles 106 and 107 is preferably an adjustable cage 110. Preferably, adjustable cage 110 is comprised of several spines 111 and two lower arms 112 that allow the cage 110 to rotate with the joint via a hinge 115 on either side of the lower arms 112. Hinge 115 may only allow for a certain range of motion (e.g. 45°, 90°, or 115°). The patient or a medical caregiver may be able to select the range of motion or the range of motion may be fixed. Spines 111 may adjust to the same range of motion. Additionally, lower arms 112 may prevent unintentional twisting or other undesirable motion of the joint. As shown in FIGS. 1 and 2, each spine 111 may be comprised of two coaxial tubes and as the patient flexes the joint (e.g. from the position in FIG. 1 to the position in FIG. 5), the inner tube may emerge from the outer tube, thereby increasing the size of cage 110. Furthermore, as the patient returns the joint to the straight position (FIG. 1), the inner tube may retract into the outer tube. Preferably, stops 113 control the coaxial movement of the tubes to prevent the tubes from becoming detached. Stops 113 may be adjustable to limit the amount of coaxial movement of the tubes. For example, the patient or a medical caregiver may adjust how far the inner tube can extend from the outer tube. In other embodiments, the spines 111 may be comprised of fabric straps, springs, elastic materials, sliding members, or other adjustable parts. Additionally, while two coaxial tubes are shown, each spine 111 may be comprised of three or more coaxial tubes. Spines 111 may be comprised of plastic, rubber, carbon fiber, fabric, metal, or another manmade or naturally occurring material. While the figures show three spines 111, fewer or more spines may be used. For example, a child's size brace 100 may have two spines, while a brace 100 for an larger patient or a patient with swollen joints may have four or more spines. Brace 100 may come in various sizes (e.g. Small, Medium, Large, Extra Large, and Extra Extra Large) to fit various sized people and joints or brace 100 may be custom fit for each patient. Preferably, the size of brace 100 is chosen or made based on the patient's body size, degree of swelling, and damage to the joint.

Preferably, attached to each lower arm 112 on either side of the hinges 115 are supports 120. Preferably, supports 120 are adapted to support the joint in an elevated position when the patient is in a prone position. Preferably, the elevated position raises the joint above the patient's heart. Supports 120 may be fixed in place or movable so that they are in a stored position when the patient is walking. Preferably, each support 120 is coupled to a lower arm 112 and a saddle 105 or 106 to provide support to the joint. However, each support 120 may be positioned at another location on the brace 100. Supports 120 may lock into the open and closed positions to avoid unintentional movement of the support during use of brace 100. Preferably, supports 120 move independently such that the patient need not use all of the supports 120 simultaneously. As shown in the embodiments of FIGS. 1-5, supports 120 may be fixed length legs that swivel from a closed position 120(A) to an open position 120(B).

Alternatively, as shown in FIG. 6, supports 620 may be pull-down or telescoping legs adjustable between a closed position 620(A) and an open position 620(B), that, when lowered to the desired length, can be locked in place, for example by twisting. The telescoping locked-in-place elevating legs may be pulled down to a length up to, for example 6 inches, 8 inches, or 10 inches. Twisting locks the supports into the desired height and untwisting allows the supports to be returned to their closed position. Similar reference numbers in FIG. 6 indicate similar elements.

A gap between the spines 111 and the lower arms 112 may provide access for the insertion of a gel pack 122 or other chilling or heating mechanism that then extends over the joint. Preferably, the gel pack 122 is held in place by the spins of the cage and clips 125 located in the corners of the cage. While two clips 125 are show, another number of clips can be used, for example 3, 4, or 6. Clips 125 may be hooks, clamps, vices, snaps, buttons, toggles, or other fastening device. Clips 125 may couple to a mating portion of gel pack 122 or may grab an edge of gel pack 122. For example, gel pack 122 may have mating snaps or holes that are engaged by clips 125. Preferably, gel pack 122 is replicable without taking off brace 100. Preferably, brace 100 is adapted to hold a variety of cooling or heating devices. However, brace 100 may have an integrated, compatible, or proprietary cooling or heating device.

Preferably, the brace 100 is worn directly on the patient's skin. A cloth or other insulating material may be placed between the gel pack 122 and the patient to temper the cooling or heating effects to avoid further injury (e.g. frostbite or burning). Alternatively, the brace 100 may be worn over tights, leggings, stockings, hose, socks, compression clothing, or other form fitting clothing. Preferably, brace 100 is worn without a cover (such as pants, long sleeves, or a jacket). For example, brace 100 may be worn with shorts, a skirt, short sleeves, a tank top, or another item of clothing. However, brace 100 may be worn with longer items of clothing. Preferably, the clothing does not interfere with the motion and utility of brace 100.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. A brace, comprising:
a first body coupling member and a second body coupling member, wherein the first and second body coupling members are adapted to surround a patient's joint;
a cage coupling the first body coupling member to the second body coupling member, wherein the cage is adapted to traverse the joint;
at least one elevating support coupled to the first body coupling member;
at least one elevating support coupled to the second body coupling member;
at least one clip coupled to the first body coupling member adapted to be coupled to a therapy device; and
at least one clip coupled to the second body coupling member adapted to be coupled to the therapy device.

2. The brace of claim 1, wherein the first and second body coupling members are saddle shaped plates securable to a patient with straps.

3. The brace of claim 2, wherein the first body coupling member is placed on a first side of the joint and the second body coupling member is placed on a second side of the joint.

4. The brace of claim 1, wherein the brace is adapted to promote the healing of a knee, an elbow, a wrist, or an ankle.

5. The brace of claim 1, wherein the cage is comprised of at least one spine and a pair of hinges.

6. The brace of claim 5, wherein each spine is comprised of at least one of two coaxial tubes or two parallel slides that accommodate movement of the patient.

7. The brace of claim 5, wherein the spines surround the therapy device.

8. The brace of claim 5, wherein the hinges permit a specified range of motion of the joint.

9. The brace of claim 1, wherein each elevating support is moveable from a closed position to an open position.

10. The brace of claim 1, wherein each elevating support is one of a swing-down arm or a telescoping arm.

11. The brace of claim 1, wherein the elevating support is one of adjustable or fixed.

12. The brace of claim 1, wherein the elevating support is adapted to raise the joint above the patient's heart while the patient is prone.

13. The brace of claim 1, wherein the therapy device is a heating device or a cooling device.

14. The brace of claim 1, wherein the patient can transition between walking and laying prone with the brace elevating the patient's joint without removing the brace.
